(19) United States
(12) Patent Application Publication
(10) Pub. No.: US 2011/0224475 A1
(43) Pub. Date: Sep. 15, 2011

(54) ROBOTIC MOBILE ANESTHESIA SYSTEM

(76) Inventors: Andries Nicolaas Schreuder, Bloomington, IN (US); John Leland Smith, Bloomington, IN (US)

(21) Appl. No.: 13/025,529

(22) Filed: Feb. 11, 2011

Related U.S. Application Data
(60) Provisional application No. 61/304,278, filed on Feb. 12, 2010.

(51) Int. Cl. A61G 15/10; A61N 5/00

(52) U.S. Cl. 600/1; 128/845

(57) ABSTRACT

A mobile anesthesia system has a patient support configured for use with a robotic patient positioning system. There is a sliding rail system disposed on the patient support and an anesthesia machine coupled to the slide rail system. Through use of the rail system(s) on the patient support a anesthesia machine may move relative to a patient support from a stored configuration clear of the patient to a treatment configuration above the patient. A method of providing anesthesia to a patient positioned to receive a robotic assisted radiation therapy is also described. The method proceeds by positioning a patient on a robotic couch in a location remote to a radiation therapy treatment room used to perform the robotic assisted radiation therapy. Next, anesthesia is administered to the patient from an anesthesia machine attached to the robotic couch while both the patient and the robotic couch are in the location remote to the radiation therapy treatment room. Thereafter, the robotic couch is placed under the control of a patient positioning system in the radiation therapy treatment room. The patient and the robotic couch are then maneuvered into a treatment position while administering anesthesia to the patient from the anesthesia machine.

### CROSS REFERENCE TO RELATED APPLICATION

This application claims the benefit under 35 U.S.C. 119 of U.S. Provisional Patent Application No. 61/304,278, filed Feb. 12, 2010, titled “Robotic Mobile Anesthesia System”, which is incorporated by reference in its entirety.

### INCORPORATION BY REFERENCE

All publications, including patents and patent applications, mentioned in this specification are herein incorporated by reference in their entirety to the same extent as if each individual publication was specifically and individually indicated to be incorporated by reference.

### BACKGROUND OF THE INVENTION

The current practice in operating rooms and intensive care units is to have an anesthesia system separate and unattached to the patient support, hospital bed, transport gurney, surgical gurney or the like. Furthermore, monitoring equipment, cables, anesthesia tubing and possibly an I.V. (intravenous fluid) set-up typically extend from the respective device to the patient on the patient support or gurney. Transporting patients from one treatment area to another requires multiple personnel and can be cumbersome. Additionally, the maneuverability of both the equipment and the medical personnel are impeded about the treatment room because of such devices.

In the instance of robotic assisted radiation therapy, it is customary to have an anesthesia system move in tandem, alongside and be separate from a patient positioner system. For background, a patient positioner system can be used to move the patient support from the mobile transport gurney to a treatment position, such as for photon, proton, electron and heavy ion radiation therapy, or the like. A patient positioner system comprises a mechanical (typically robotic) support system that moves along multiple translational and rotational axes within a gantry for radiation to align the patient in proper position for receiving the radiation beams.

The equipment arrangement in the treatment room with an independent anesthesia and monitoring system leads to possible conflict in patient positioner movement. This limits the range of motion for the robotic patient positioner system (“PPS”), all the while simply adding more equipment to the treatment room.

Another direct consequence of the existing practice outlined above has to do with the duration of time that the treatment room is being occupied. In any medical setting, the duration of occupancy of a treatment room is extended when a patient is anesthetized in the treatment room before the treatment procedure commences and similarly when the patient is recovered from the anesthesia in the treatment room after the procedure is completed. In the instance of radiation beam therapy systems that have multiple treatment rooms and share resources such as the radiation source, the duration of occupancy in a treatment room is of great importance. Ultimately it impacts the system workflow and efficiency of the patient throughput.

In addition, another disadvantage of the current practice in treatment rooms (both for robotic assisted radiation therapy and other medical treatment rooms) involves the overall patient experience. Irrespective of the treatment procedure, patients can become scared, unable to relax and experience heightened anxiety as part of undergoing treatment.

For the various reasons discussed above and not limited thereto, an improved system for transporting and treating anesthetized patients in a treatment environment is needed that will contribute to patient workflow efficiencies, improve safety and maneuverability within the treatment room, as well as be beneficial to the patient experience.

### SUMMARY OF THE INVENTION

A mobile anesthesia system is provided that overcomes the disadvantages in moving patients requiring anesthesia and meets the above objectives in medical environments, more specifically radiation beam therapy systems. In some embodiments, the mobile anesthesia system can include a safe and effective PPS that upholds a patient support couch with a rail system to support an anesthesia and patient monitoring system. The patient support couch can be configured with a frame affixed to a rail system which the anesthesia machine and associated equipment slide along. The rail system provides for movement along the length of the couch from a stowed position beyond the foot of the patient to a temporary, forward position over the body of the patient and within reach of the anesthesiologist. The rail system can also include a treatment position where the system is in use but outside of the zone of treatment (i.e., does not interfere with treatment).

Some embodiments include a safe and secure means of getting a patient into position for receiving treatment while optimizing the maneuverability of the equipment and the personnel about the treatment room. Furthermore, the ability to anesthetize and possibly also recover a patient from the anesthesia in a comfortable and less intimidating environment than the treatment room would help improve the patient's treatment experience as well as reduce the occupancy time of the treatment room.

In an additional aspect, there is provided mobile anesthesia system having a patient support configured for use with a robotic patient positioning system; a sliding rail system disposed on the patient support; and an anesthesia machine coupled to the slide rail system and configured to move along the rail system from a stored configuration to a treatment configuration.

In one alternative embodiment, the patient support is a robotic couch. Still further, the robotic couch is divided into a treatment zone and an anesthesia machine support zone and the sliding rail system is disposed completely outside of the treatment zone. The patent treatment zone may include a thermoplastic support and support zone may include a non-thermoplastic or a metal support plate. The patient support may also comprise a carbon fiber material that is radiolucent to the radiation used to treat the patient. The sliding rail system is positioned on the patent support no as to not interfere with the patient treatment. In addition, the material used to fabricate the robotic couch in the treatment zone is selected to reduce interference with a treatment procedure performed on the patient supported by the robotic couch and the material used to fabricate the robotic couch in the anesthesia machine support zone is selected without regard to interference with a treatment procedure performed on the patient supported by the robotic couch.

In additional aspects, the sliding rail system includes a pair of rails arranged one each on opposite sides of the patient support. Alternatively, the rail system has a single rail on one side of the patient support. In another variation, the sliding rail system is configured to allow horizontal movement of the anesthesia machine relative to the patient support. In another alternative, there is a horizontal rail system and then a second sliding rail system oriented relative to the patient support for vertical movement of the anesthesia machine relative to the patient support. The support rail or rails may be mechanically or motor assisted as well as provided with locking mechanisms to the anesthesia machine in one or both of a horizontal and vertical position relative to the patient or patient support.

There may also be provided a pivoting arm connected between the anesthesia machine and the sliding rail system or a support arm attached to the rail system. In one aspect, the anesthesia machine moves along the rail system when transitioning from a stored configuration to a treatment configuration. The anesthesia machine is positioned near a first end of the patient support in the stored configuration and is positioned near a second end of the patient support in the treatment configuration. Still further, there may also be provided a foot support positioned on the patient support to separate the patient near the first end from the anesthesia machine near the second end. In one alternative, the first end is beyond the feet of a patient on the patient support and the second end is above a patient on the patient support. The first end may also be beyond the patient's head or at the distal end of a patient support surface.

In one embodiment, there is a strut extending between patient support and the anesthesia machine to maintain the position of the anesthesia machine above the patient. The strut may also include any of a wide variety of mechanisms to support the weight of the anesthesia machine as well as provide for an adjustable height above the patient. In one embodiment, the strut includes a hydraulic ram coupled at one end to the anesthesia machine and at another end to the patient support.

In one embodiment, the anesthesia system also includes a portable gas system in communication with the anesthesia machine. The portable gas system moves along with the patient support when the patient is positioned for a therapy and remains in position relative to the patient support while the patient is receiving therapy. There is also provided a suitable gas switch or valve bank that permits a user to align the anesthesia machine to receive treatment gas or gases from the portable tanks on the anesthesia machine or from a supply of gases, such as from a medical facility where a patient is being treated. Still further, the anesthesia machine may also include a switching mechanism configured to switch the gas supply for the anesthesia machine between the portable gas system and a medical facility gas system.

In additional aspects, there is provided a method of providing anesthesia to a patient positioned to receive a robotic assisted radiation therapy. The method of providing anesthesia proceeds by positioning a patient on a robotic couch in a location remote to a radiation therapy treatment room used to perform the robotic assisted radiation therapy; administering anesthesia to the patient from an anesthesia machine attached to the robotic couch while the patient and the robotic couch are in the location remote to the radiation therapy treatment room; placing the robotic couch under the control of a patient positioning system in the radiation therapy treatment room; and maneuvering the patient and the robotic couch into a treatment position while administering anesthesia to the patient from the anesthesia machine.

There may also be the added step of using a portable gas system in communication with the anesthesia machine during the placing step and the maneuvering step. Still further, there may also be a step of switching an anesthesia machine gas source between a portable gas system and a medical facility gas system while performing the administering step. One alternative step may also include moving the anesthesia machine relative to a portion of the robotic couch using a sliding rail system coupled to the robotic couch. In additional embodiments, there may be the step of moving the anesthesia machine from a stowed configuration to a treatment configuration before the administering step. Another step may include a method where the administering step also includes supplying gas to the anesthesia machine from a portable gas system that moves along with the robotic couch while the robotic couch is under the control of a patient positioning system. There is also the step of transferring the patient support from a transport gurney to the robotic couch. This transferring step occurs during the administering step, in some aspects. Still further, the method may include transporting the patient from the remote location to a treatment location. In addition, the method may include delivering a robotic assisted radiation therapy to the patient, including treatment of the patient with a proton beam. Still further, the transporting step may also include maintaining the anesthesia machine in a stowed position on the robotic couch while transporting the patient from a position remote to a radiation therapy treatment room to the radiation therapy room. There may also be the additional step of moving the anesthesia machine from a stowed condition to a treatment condition above the patient while performing the administering step.

### BRIEF DESCRIPTION OF THE DRAWINGS

These and various other features and advantages of the present invention will become better understood as the following detailed description is studied in conjunction with the accompanying drawings, where:

FIGS. 1A-1B are isometric views of a mobile anesthesia system (without a transport gurney) shown in the stowed configuration in FIG. 1A and in a treatment configuration in FIG. 1B.

FIG. 2A is an additional views of a mobile anesthesia system, having a single rail system and a pivoting arm connecting between the anesthesia machine and the sliding rail system.

FIG. 2B is an isometric, bottom up view of the distal end of the rail system showing the attachment to the patient support.

FIG. 3 is a view of a mobile anesthesia machine decoupled from the patient support and coupled to a portable anesthesia cart.

FIG. 4 is an isometric view of a treatment room with a mobile anesthesia system receiving anesthetic gases from a medical facility.

FIGS. 5A-5B are close in and isometric views, respectively, of a locking mechanism for positioning the mobile anesthesia system.

FIG. 6 illustrates an isometric view of a patient gurney relative to a camera based guidance system in the floor of a treatment room.

FIG. 7 is a schematic diagram illustrating one configuration of the mobile anesthesia system in a treatment room supported by a robotic patient positioning system in a radiation therapy treatment room.

### DETAILED DESCRIPTION OF THE INVENTION

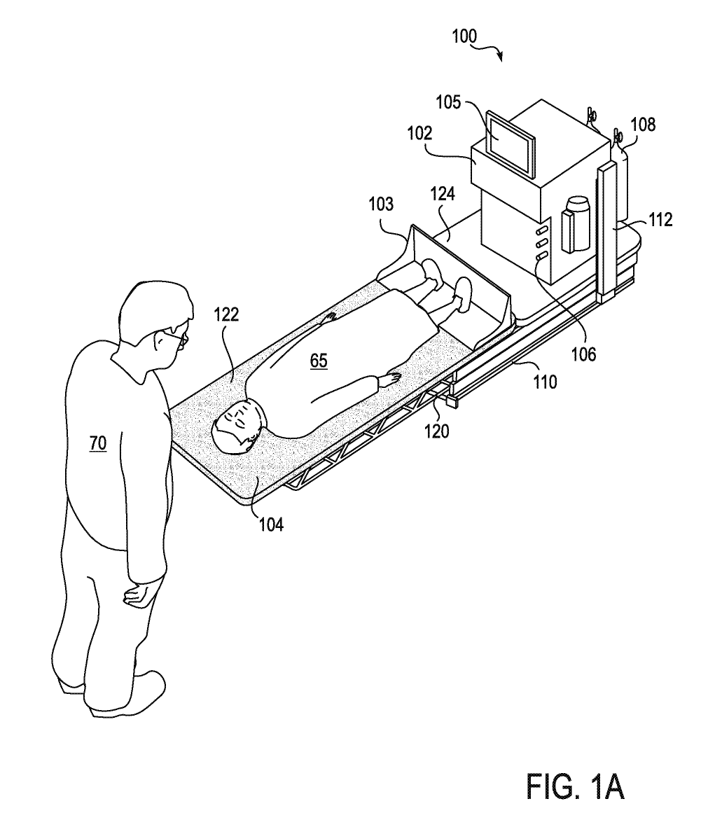

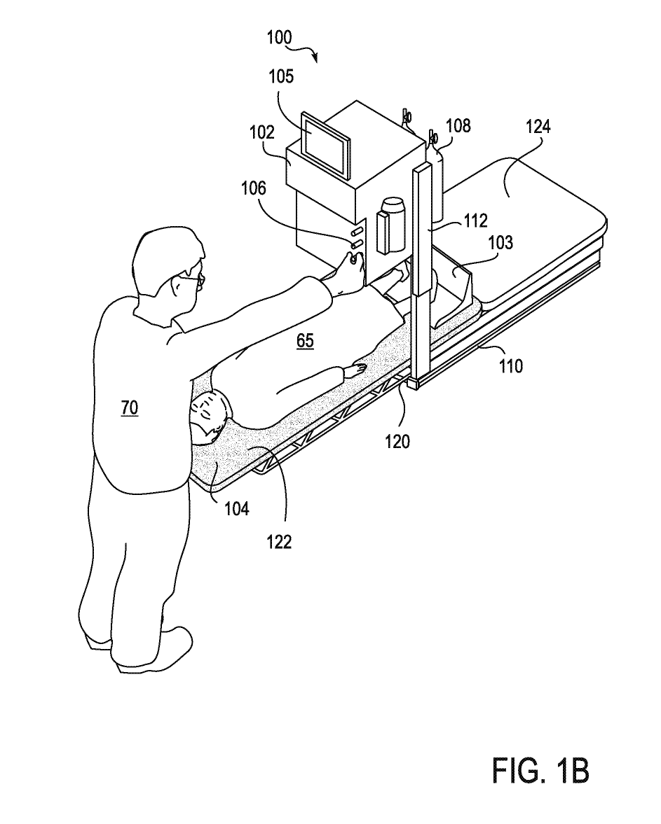

FIGS. 1A-1B illustrate a mobile anesthesia system 100, including an anesthesia machine 102 and a patient support 104. The patient support can rest upon a hospital bed, transport gurney, or the like (not shown). The patient support 104 includes a support frame 120 that supports a patient support 122 and an equipment support 124. The support frame 120 also bears the weight of and provides mechanical support and attachment for the rail system 110. The patient support 122 may be made from any suitable material for the type of therapy being administered to the patients. The patient support 122 may be formed from a thermoplastic, a carbon fiber or other suitable material. In one aspect the patient support 122 is a radio translucent material. The equipment support 124 may be formed from a material different from the patient support 122 such as aluminum, metal alloy or a non-thermoplastic material. The equipment support 124 is used to provide additional mechanical strength to the patient support 104 particularly around rail system 110. In one aspect, the equipment support 124 corresponds to the length of the rail system 110. The equipment support 124 and the patient support 122 may meet end to end or may overlap.

The anesthesia machine 102 can comprise controls 106 for the administration of anesthesia to a patient, and a portable gas system 108 configured to allow administration of anesthesia to the patient during transport as well as before, during and after treatment. The portable gas system 108 can include cylinders of essential anesthetic gases, including oxygen, medical air, nitrous oxide, for example. The portable gas system can be affixed to mobile anesthesia system in various ways, such as directly affixed to the anesthesia machine or to the patent support. The portable gas system can also be part of an independent means of transporting or delivering the anesthetic gases with the anesthesia machine. The anesthesia machine can further include any number of features typically found in anesthesia machines, including a power supply, reserve gas cylinders, flow meters, pressure gauges, vaporizers, ventilators, physiological monitors (e.g., heart rate, ECG, blood pressure, oxygen saturation, etc), breathing circuits, heat exchangers, and suction, for example. Other and further modifications may include incorporating poles and hooks to securely support or stow any additional items necessary for the treatment, such as fluids or medications.

In another embodiment, a foot support 103 may be included which can provide a guide for ensuring the clearance of the patient's feet by the anesthesia machine. While this can serve as an added safety measure, the foot support can be sized so as to not obstruct the view of the medical practitioners. In other embodiments, the foot support can be latticed or transparent so as to not obstruct the view of the medical practitioners. In another embodiment of a mobile anesthesia system, a monitor 105 can be included as part of the mobile anesthesia system for patient monitoring purposes. The monitor 105 can be mounted to provide for the capability of pivoting for adjustment of the position and viewing angle of the monitor.

In some embodiments, the mobile anesthesia system can further comprise a rail system 110 disposed on the patient support. As shown in FIGS. 1A-1B, the anesthesia machine 102 is coupled to two support arms 112 attached to the rail system 110 on opposite sides of the patient support. Only one side of the rail system 110 is visible on this view. The anesthesia machine 102 is configured to move from a stowed configuration, as shown in FIG. 1A, to a treatment configuration (also referred to as a anesthesia preparation (“prep”) position), as shown in FIG. 1B. The arms 112 and anesthesia machine 102 slide along and are supported by the rail system 110. In the embodiment of FIG. 1A, the anesthesia machine is positioned in the stowed position i.e. just above the patient support and near the patient's feet at the tail end of the patient support 104. this position, the anesthesia's machine 102 is supported by the equipment support 124. The anesthesia machine 102 can be positioned in the stowed configuration during transport or prior to the patient receiving anesthesia, for example. When the anesthesia machine 102 is positioned in the stowed configuration, the anesthesia machine does not interfere with a patient lying on the patient support. As a safety feature, the anesthesia machine cannot move forward while in the stowed position, as shown in FIG. 1A. In the embodiment of FIG. 1B, the anesthesia machine 102 is positioned in the anesthesia prep position i.e. above the patient 65. When the anesthesia machine positioned in the anesthesia prep position, the anesthesia machine is easily accessible by a medical practitioner 70, such as an anesthesiologist. More specifically, if the anesthesiologist 70 is standing near the head of the patient 65 resting on the patient support 122, the anesthesia prep position allows the medical practitioner 70 to easily reach the controls 106 of the anesthesia machine 102. In other words, the rail system 110 extends along the patient support frame 120 to permit positions above the patient.

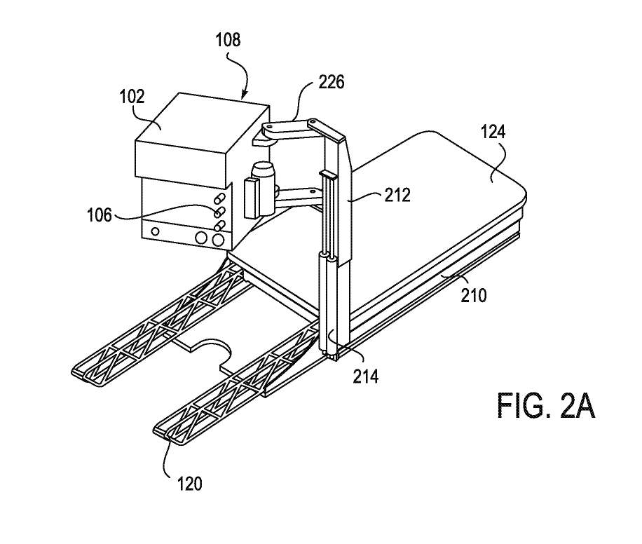

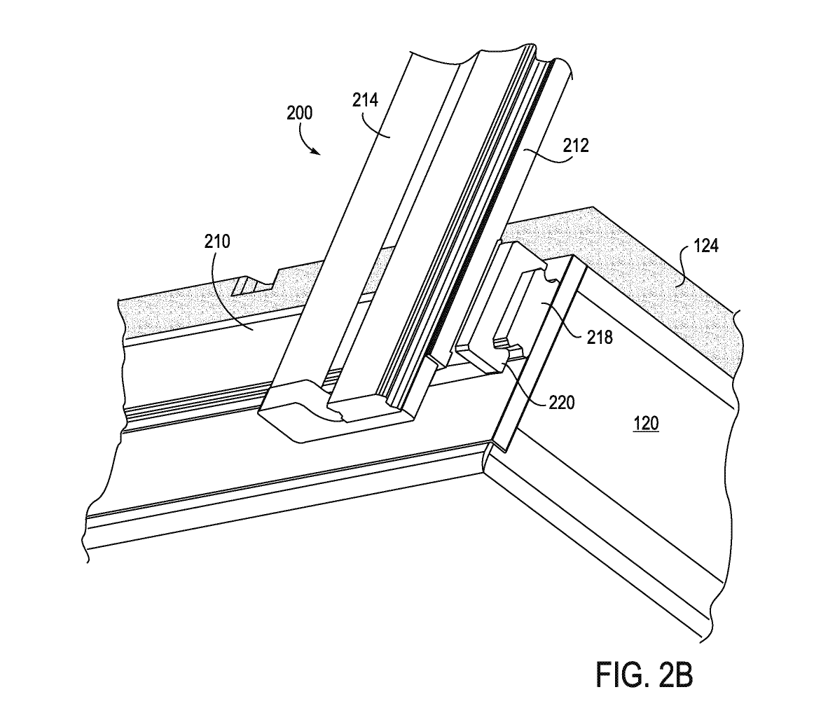

FIG. 2 is an alternative to the mobile anesthesia system described above, including anesthesia machine 102, patient support 104, single sided rail system 210, single support arm 212, and strut 214. Referring still to FIG. 2A, it can be seen that anesthesia machine 102 is coupled to rail system 210 via support arm 212 and reinforced by strut 214. Single side rail system 210 is supported by support frame 120 and configured to allow horizontal movement of the anesthesia machine 102 relative to the patient support 104. Similarly, a vertical rail system and strut 214 are configured to allow vertical movement of the anesthesia machine 102 relative to the patient support 104. The anesthesia machine 102 is in the elevated position when it is above the patient in the forward, anesthesia prep position (see FIG. 2A). This is an important aspect of the anesthesia prep position to prevent the anesthesia machine from falling down on top of the patient. One refinement includes the ability of the strut 214 to counterweight the anesthesia machine, requiring a downward force to lower the anesthesia machine from a raised position to a lowered position. The strut 214 illustrated in FIG. 2A is a hydraulic ram that supports the weight of the anesthesia machine 102 when elevated. The two arm system shown in FIGS. 1A and 1B may be modified to include a support such as strut 4, or other suitable counter weight.

In the embodiments of FIGS. 1A-1B, the rail systems 110 can be disposed on two sides of the patient support 104. However, in other embodiments, the rail system can be disposed on only one side of the patient support 104, as shown in FIG. 2A. In these embodiments, the anesthesia machine 102 may be mounted to the support frame by a cantilever arm or arms. The arms can further include swivel joints to allow for re-positioning of the anesthesia machine. In the illustrated embodiment, the anesthesia machine 102 is supported by arms 212 and 226. A pivot or hinge is provided on arm 226.

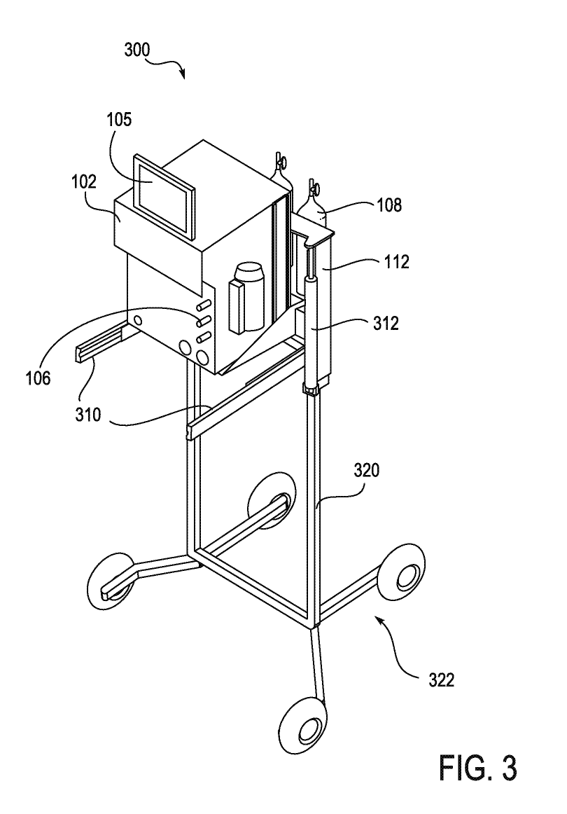

FIG. 2B, is a close up of the rail system 210. The rail system may include safety stops 220, which prevents the anesthesia machine 102 and arm 112 from sliding off of the rail system 210 and disconnecting or falling from the patient support. In some embodiments, the safety stops may be mechanical, adjustable, removable, or a combination thereof to allow the anesthesia machine to be detached from the patient support for transfer onto a different storage location as a mobile cart as shown in FIG. 3.

Referring to FIG. 2B, single side rail system 210 includes a rail 218 and a sliding mechanism configured and sized to couple to and slide along the rail 218. The rail 218 can include a groove and the sliding mechanism can be shaped and configured to rest in and slide along the groove, as shown. In other embodiments, the rail system can comprise any rail system as known in the art, including a rail adhesion system, a rack and pinion system, or a grooved wheel rail system, for example. The support arm 212 can also comprise any of the rail systems described herein or known in the art. The rail system 110 and arm 112 are similarly configured. In summary, the rail extends along the patient support and the arm couples to the rail so as to slide along the rail while supporting the anesthesia machine 102.

FIG. 3 illustrates one embodiment of a portable anesthesia cart 300 that may be used with the mobile anesthesia systems described above. In FIG. 3, anesthesia system 102 is shown decoupled from a patient support, such as the patient supports shown in FIGS. 1A-1B and 2A. The portable anesthesia cart 300 can include rails 310 sized and configured to mate with rails of the dual rail system 110 described above and may be modified for rail system 210. The rails 310 are supported by a frame 320 on a wheeled based 322. The rails 310 may be at a fixed height or provided with an adjustment to alter the height of the rails 310 above the wheeled base. An adjustment system permits the height the rails 310 to vary so that the portable cart 300 may be used with patient supports positioned at different heights.

To load the anesthesia system 102 from the patient support to the portable anesthesia cart 300, the rails 310 of the portable anesthesia cart can be aligned with the rails of the patient support (i.e., the end of rail system). If the patient support includes a safety stop, the safety stop can be removed or adjusted to accommodate for offloading the anesthesia machine 102 from the patient support 104 as previously described. In some embodiments, multiple anesthesia machines, such as portable or fixed anesthesia systems, can be incorporated or loaded onto a single portable anesthesia cart. FIG. 3 also illustrates a two arm support (similar to FIG. A) that includes a strut 312 to support and/or adjust the vertical height of the anesthesia machine 102 along arm 112.

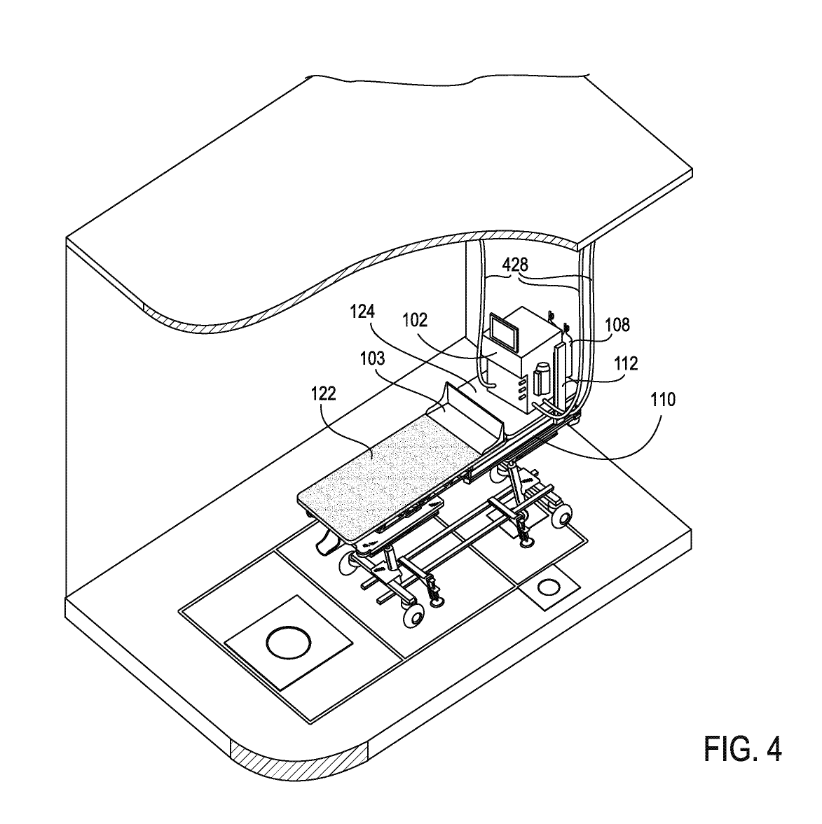

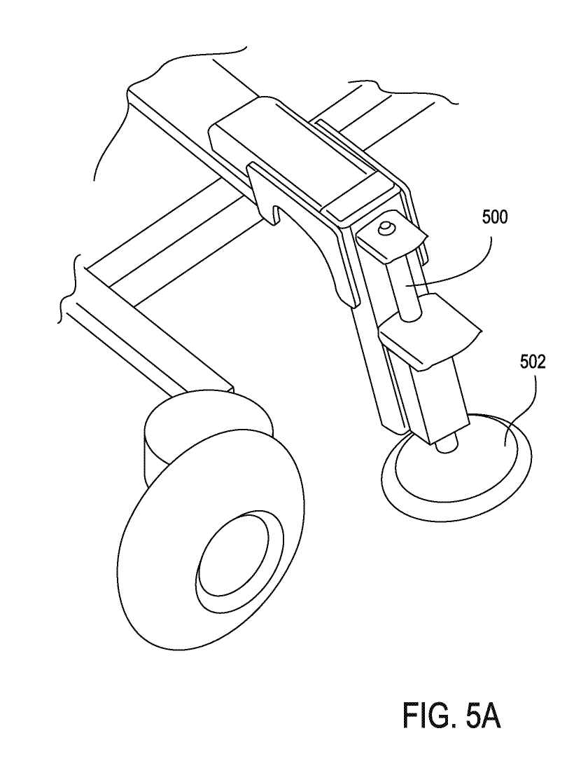

In FIG. 4, a mobile anesthesia system is shown hooked up to a medical facility gas system via gas supply lines 428. The mobile anesthesia system can include a switching mechanism (not shown) configured to switch between the portable gas system)08 and the medical facility gas system. The ability to switch between a fixed supply of anesthetic gases provided through the medical facility gas system and the portable gas system provides versatility with respect to the location where the patient undergoes anesthesia. For example, an anesthesiologist can administer anesthesia to a patient using a portable gas system 108 during patient transport, or when the patient is outside a treatment room. When the patient is taken into the treatment room, the mobile anesthesia system can then be hooked up to the medical facility gas system and switched off of the portable gas system. The patient may continue to receive anesthesia during the switch from the portable gas system to the medical facility gas system.

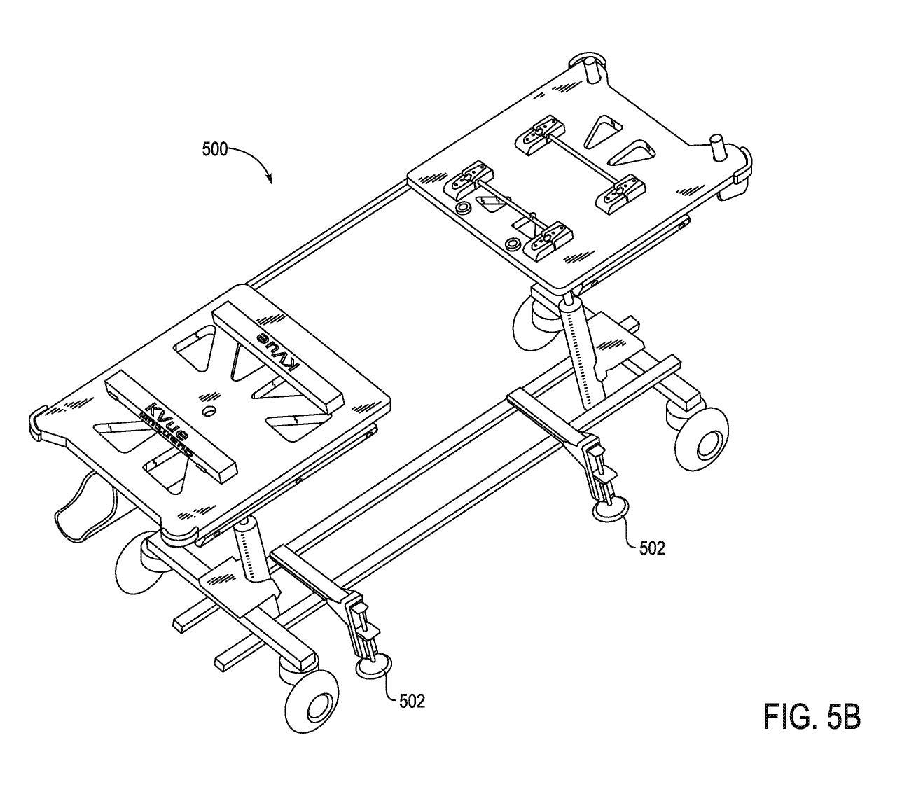

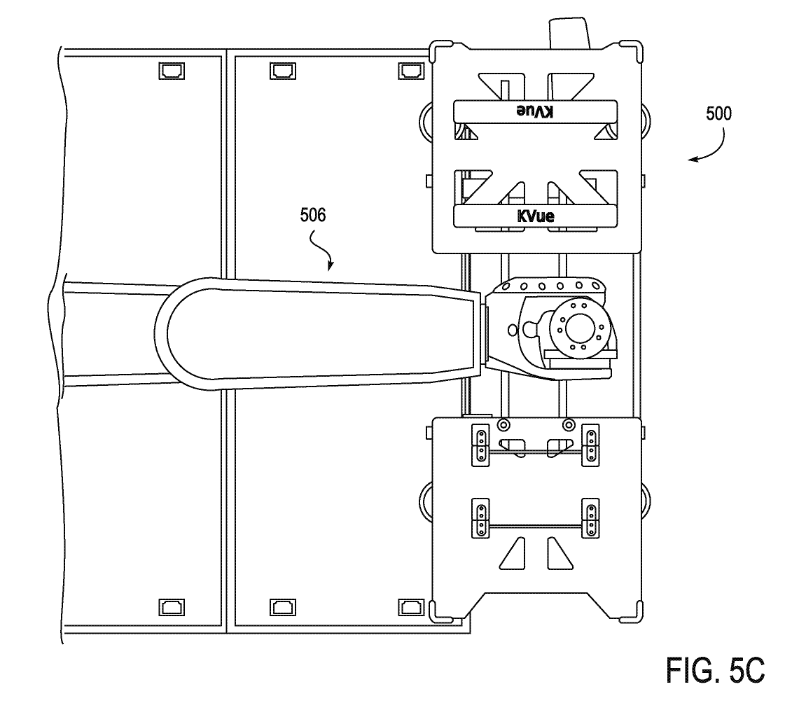

FIGS. 5A-5C illustrate a locking mechanism 502 for positioning the transport gurney carrying a mobile anesthesia system (not shown). When the transport gurney 500 with the patient support and mobile anesthesia system are ready to be received by the PPS 506, a locking mechanism 502 can be employed to stabilize the position of the patient and transport gurney, as shown in FIG. 5B. The PPS 506 can then properly align without any personnel necessary to hold the transport gurney still, as shown in FIG. 5C. The more objects or people surrounding the PPS and the transport gurney, the greater the limitation on the range of movement of the PPS.

Once the transport gurney is locked into place, the PPS 506 can then mechanically retrieve the patient support from the transport gurney and then move the patient into the proper position to receive the treatment. Conversely, when the patient's treatment is complete, the PPS can then replace the patient support back onto the transport gurney. Further details on the interaction between a PPS and a patient support are described in U.S. patent application Ser. No. 12/208,852, titled “Imaging Positioning System Having Robotically Positioned D-Arm”, filed on Sep. 11, 2008. The mobile anesthesia system is a truly unique solution, particularly in robotic assisted radiation therapy, for the ability to administer anesthesia to a patient before, during and after treatment without interruption and reducing potential for interference of peripheral equipment with the movement and positioning of the patient positioner during treatment.

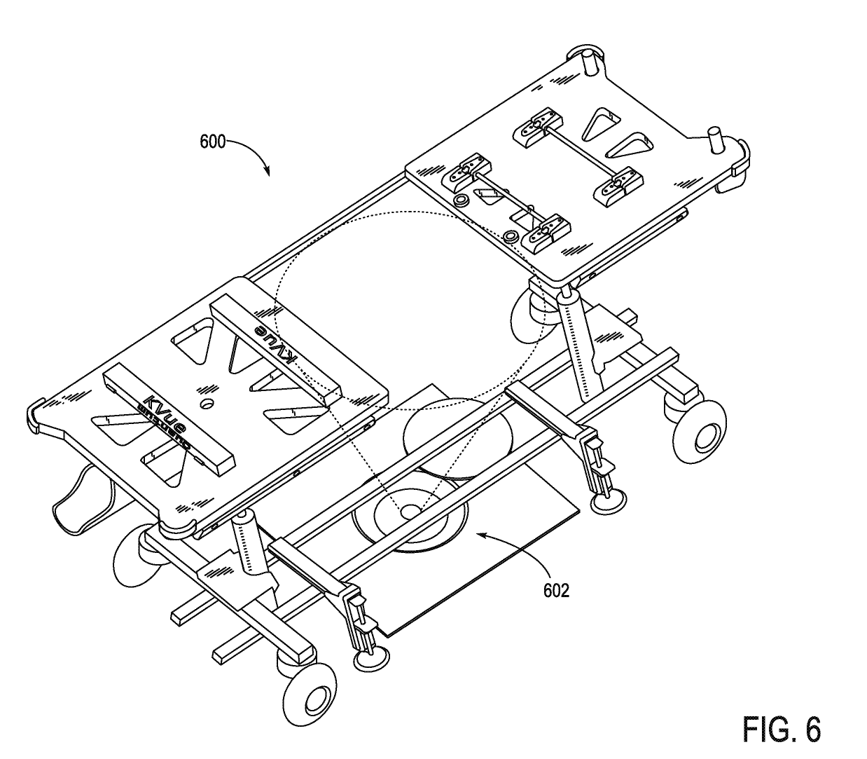

In the embodiment of FIG. 6, a camera based guidance system 602 can be employed to help align the PPS in the proper position. The PPS can utilize the assistance of a camera based system to guide it into the proper position for retrieving the patient support from the transport gurney 600. In one embodiment, the camera based system would be affixed in the floor of the treatment room in order to facilitate the alignment process from underneath the patient support.

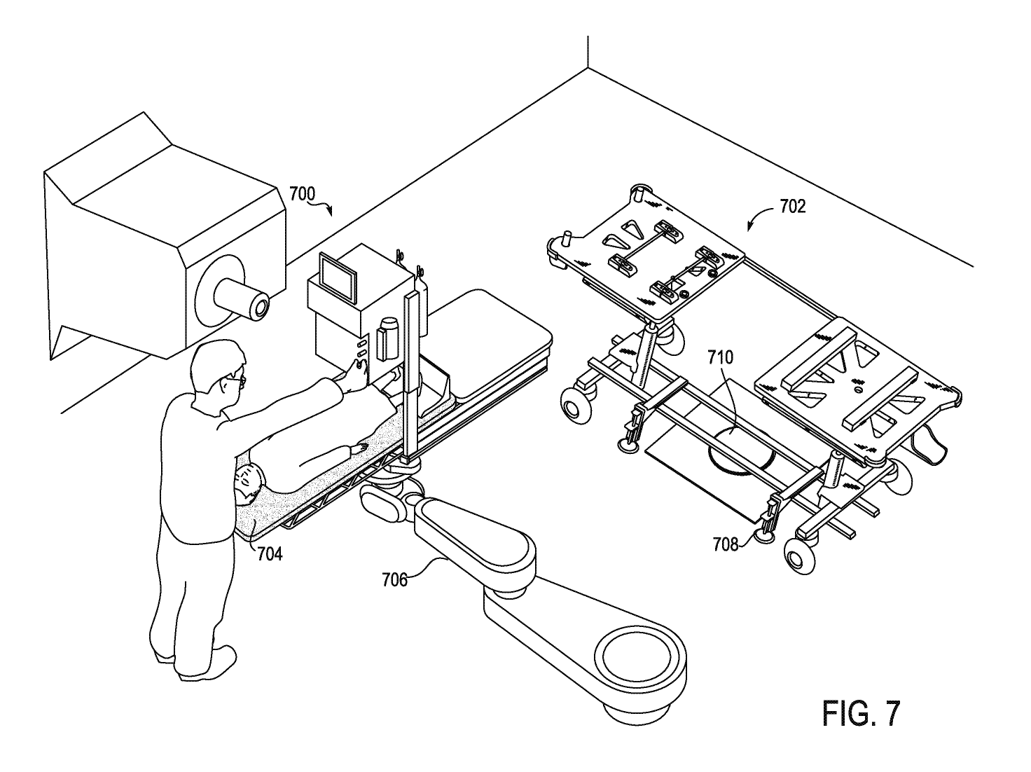

FIG. 7 is a diagram illustrating one configuration of a mobile anesthesia system 100 in a treatment room. With the use of the mobile anesthesia system 100, the PPS and personnel have fewer limitations on the range of movement in the treatment room. An added benefit is that the anesthesia machine 102 can be attached to the patient support 104 so that all features of the anesthesia machine (such as pipes, gas containers, tubes, wires, etc) are contained within the patient support. Therefore, the anesthesia machine 102 and its features are configured to move with the patient support 104 so that nothing drags on the floor or interferes with the environment, the physician, or the patient.

This mobile anesthesia system can also be used in the normal operating room (“OR”) when equipped with any equivalent patient transport and positioning system known in the art. The advantages for normal operating room treatments would be numerous, including the ability to anesthetize an anxious patient in a calm, comfortable environment other than the operating room then transport the patient to the treatment room. Similarly, if a patient were to arrive into the emergency room (“ER”) with a broken bone, for example, anesthesia could be administered to the patient before being transported to the treatment room for setting the fractured bone.

Methods of treating a patient are also provided. The methods can use any of the mobile anesthesia systems described herein. In one embodiment, a method of treating a patient comprises positioning a patient on a patient support, and administering anesthesia to the patient with an anesthesia machine to the patient support.

The method can further comprise the step of moving the anesthesia machine from a stowed configuration to anesthesia prep position before the step of administering anesthesia to the patient. In some embodiments, the rail system is configured to allow only a horizontal movement of the anesthesia machine along the patient support. In other embodiments, the rail system is further configured to allow first a vertical movement of the anesthesia machine and then a horizontal movement to position the anesthesia equipment above and in close proximity to the clinician (see FIG. 1B). As described above, the anesthesia machine and its features are configured to be contained within the patient support so as to move during transportation of the patient support without interfering with the environment, the physician, or the patient.

In some embodiments, the method comprises administering anesthesia to the patient in a remote location separate from a treatment room. The treatment room can be a location configured for photon, proton, electron and heavy ion radiation therapy, or the like. The remote location can be, for example, a waiting room, a recovery room, an emergency room, or a preparation room separate from the treatment room. The method can further comprise the step of transporting the patient from the remote location to the treatment room.

The administering anesthesia step can further comprise administering anesthesia to the patient with a portable gas system. In some embodiments, the method can further comprise the step of switching from the portable gas system to a medical facility gas system. It should be noted that when the anesthesia machine is hooked up to the medical facility gas system, there may be wires or tubes extending away from the anesthesia machine and patient support. However, these components can be connected to the anesthesia machine so as not to interfere with the physician, the patient, moving the anesthesia machine between the stowed position and the anesthesia prep position, or with moving the patient support between various treatment positions. Anesthesia can continue to be administered to the patient with the medical facility gas system after the switching step. In sonic embodiments, anesthesia can be administered to the patient during the switching step.

The method can further comprise the step of transferring the patient support from a transport gurney to a robotic couch. In some embodiments, the transferring step occurs before, during or after the administering anesthesia step. The anesthesia machine moves with the patient support during the transferring step. As described above, all features of the anesthesia machine are contained within the patient support, and thus all features will move with the patient support during the transferring step without interfering with the environment, the physician, or the patient.

In yet another embodiment, the method comprises the step of delivering proton or radiation therapy to the patient. The proton or radiation therapy can be delivered to the patient while the patient is being administered anesthesia.

Additional details of the gurney illustrated and described in, for example, FIGS. 4, 5A, 5B, 6 and 7 are provided in copending, commonly assigned non-provisional patent application Ser. No. __/___,___ by Andries Nicolaas Schreuder, filed Feb. 11, 2011, titled “PATIENT GURNEY HAVING CONFIGURABLE REGISTRATION CAPABILITIES”, the entirety of which is incorporated herein by reference.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A mobile anesthesia system, comprising:
a patient support configured for use with a robotic patient positioning system;
a sliding rail system disposed on the patient support; and
an anesthesia machine coupled to the slide rail system and configured to move along the rail system from a stored configuration to a treatment configuration.

2. The anesthesia system of claim 1 wherein the patient support is a robotic couch.

3. The anesthesia system of claim 2, wherein the robotic couch is divided into a treatment zone and an anesthesia machine support zone and the sliding rail system is disposed completely outside of the treatment zone.

4. The anesthesia system of claim 3, wherein the material used to fabricate the robotic couch in the treatment zone is selected to reduce interference with a treatment procedure performed on the patient supported by the robotic couch and the material used to fabricate the robotic couch in the anesthesia machine support zone is selected without regard to interference with a treatment procedure performed on the patient supported by the robotic couch.

5. The anesthesia system of claim 1, the sliding rail system further comprising: a pair of rails arranged one each on opposite sides of the patient support.

6. The anesthesia system of claim 1, the rail system further comprising: a single rail on one side of the patient support.

7. The anesthesia system of claim 1 further comprising: a pivoting arm connected between the anesthesia machine and the sliding rail system.

8. The anesthesia system of claim 1 wherein the anesthesia machine moves along the rail system when transitioning from a stored configuration to a treatment configuration.

9. The anesthesia system of claim 8 wherein the anesthesia machine is positioned near a first end of the patient support in the stored configuration and is positioned near a second end of the patient support in the treatment configuration.

10. The anesthesia system of claim 9 further comprising: a foot support positioned on the patient support to separate the patient near the first end from the anesthesia machine near the second end.

11. The anesthesia machine of claim 9 wherein the first end is beyond the feet of a patient on the patient support and the second end is above a patient on the patient support.

12. The anesthesia system of claim 1 wherein the sliding rail system is configured to allow horizontal movement of the anesthesia machine relative to the patient support.

13. The anesthesia system of claim 1 further comprising: a second sliding rail system, the second sliding rail system oriented relative to the patient support for vertical movement of the anesthesia machine relative to the patient support.

14. The anesthesia system of claim 13 further comprising: a strut extending between patient support and the anesthesia machine to maintain the position of the anesthesia machine above the patient.

15. The anesthesia system of claim 14 wherein the strut comprises a hydraulic ram coupled at one end to the anesthesia machine and at another end to the patient support.

16. The anesthesia system of claim 1 further comprising: a portable gas system in communication with the anesthesia machine.

17. The anesthesia system of claim 16 wherein the portable gas system moves along with the patient support when the patient is positioned for a therapy and remains in position relative to the patient support while the patient is receiving therapy.

18. The anesthesia system of claim 16 the anesthesia machine further comprising: a switching mechanism configured to switch the gas supply for the anesthesia machine between the portable gas system and a medical facility gas system.

19. A method of providing anesthesia to a patient positioned to receive a robotic assisted radiation therapy, comprising:
positioning a patient on a robotic couch in a location remote to a radiation therapy treatment room used to perform the robotic assisted radiation therapy; administering anesthesia to the patient from an anesthesia machine attached to the robotic couch while the patient and the robotic couch are in the location remote to the radiation therapy treatment room;
placing the robotic couch under the control of a patient positioning system in the radiation therapy treatment room;
maneuvering the patient and the robotic couch into a treatment position while administering anesthesia to the patient from the anesthesia machine.

20. The method of claim 19 further comprising: performing the administering step using a portable gas system in communication with the anesthesia machine during the placing step and the maneuvering step.

21. The method of claim 19 further comprising: switching an anesthesia machine gas source between a portable gas system and a medical facility gas system while performing the administering step.

22. The method of claim 19 further comprising: moving the anesthesia machine relative to a portion of the robotic couch using a sliding rail system coupled to the robotic couch.

23. The method of claim 22 the moving step further comprising: moving the anesthesia machine from a stowed configuration to a treatment configuration before the administering step.

24. The method of claim 19 the administering step further comprising: supplying gas to the anesthesia machine from a portable gas system that moves along with the robotic couch while the robotic couch is under the control of a patient positioning system.

25. The method of claim 19 further comprising the step of transferring the patient support from a transport gurney to the robotic couch.

26. The method of claim 25 wherein the transferring step occurs during the administering step.

27. The method of claim 19 further comprising: transporting the patient from the remote location to a treatment location.

28. The method of claim 19 further comprising: delivering a robotic assisted radiation therapy to the patient.

29. The method of claim 27 the transporting step further comprising: maintaining the anesthesia machine in a stowed position on the robotic couch while transporting the patient from a position remote to a radiation therapy treatment room to the radiation therapy room.

30. The method of claim 19 further comprising: moving the anesthesia machine from a stowed condition to a treatment condition above the patient while performing the administering step.
